(19) United States
(12) Patent Application Publication
(10) Pub. No.: US 2003/0099691 A1
(43) Pub. Date: May 29, 2003

(54) Films containing starch

(76) Inventors: Susan Lydzinski, Belle Mead, NJ (US); Todd Manegold, Somerset, NJ (US); Daniel B. Solarek, Hillsborough, NJ (US); John J. Tsai, Belle Mead, NJ (US); Christine Puglisi, Mountainside, NJ (US)

Correspondence Address: Karen G. Kaiser, NATIONAL STARCH AND CHEMICAL COMPANY, 10 Finderne Avenue, Bridgewater, NJ, 08807-0500, US

(21) Appl. No.: 09/991,385

(22) Filed: Nov. 16, 2001

(51) Int. Cl. A61K 9/70; A61K 7/16; A61K 31/718

(52) U.S. Cl. 424/443; 514/60

(57) ABSTRACT

The present invention relates to a film, particularly an oral film, which contains starch as the main component. Such film is useful for delivering a variety of agents to humans and other animals to produce a therapeutic, organoleptic, or cosmetic effect, including breath fresheners, flavors, fragrances and pharmaceuticals.

### BACKGROUND OF THE INVENTION

The present invention relates to a film, particularly an oral film, which contains starch as the main component. Such film is useful for delivering a variety of agents to humans and other animals.

There are a variety of agents which can be delivered to produce a therapeutic, organoleptic, or cosmetic effect, including breath fresheners, flavors, fragrances and pharmaceuticals. Such agents are typically delivered in a number of ways including, mouth washes and sprays, tablets, chewing gums, and transdermal patches. For example, U.S. Pat. No. 6,197,288 discloses a method of counteracting of a malodor in the oral cavity by delivering a composition orally.

Recently, a new method of delivering such agents has been disclosed, that of delivering by oral film. For example, U.S. Pat. No. 6,153,222 discloses a volume-expandable, sheet-like form suitable to carry an active agent and U.S. Pat. No. 6,177,096 discloses a composition comprising a water-soluble polymer, at least one polyalcohol, and at least one cosmetically or pharmaceutically active ingredients, wherein the composition has mucoadhesive properties.

Many of the marketed films contain pullulan as the main component. However, pullulan is expensive to manufacture and import. Therefore, others polymers have been substituted for pullulan, including native starch. Native starch has been found to be inferior to pullulan in functionality, particularly in that it forms brittle films.

Surprisingly, it has now been found that modified starches, particularly stabilized starches, form excellent films useful for delivering a wide variety of actives.

### SUMMARY OF THE INVENTION

The present invention relates to a film, particularly an oral film, which contains starch as the main component. Such film is useful for delivering a variety of agents to humans and other animals to produce a therapeutic, organoleptic, or cosmetic effect, including breath fresheners, flavors, fragrances and pharmaceuticals.

### DETAILED DESCRIPTION OF THE INVENTION

The present invention relates to a film, particularly an oral film, which contains starch as the main component. Such film is useful for delivering a variety of agents to humans and other animals to produce a therapeutic, organoleptic, or cosmetic effect, including breath fresheners, flavors, fragrances and pharmaceuticals.

Starch, as used herein, is intended to include all starches derived from any native source, any of which may be suitable for use herein. A native starch as used herein, is one as it is found in nature. Also suitable are starches derived from a plant obtained by standard breeding techniques including crossbreeding, translocation, inversion, transformation or any other method of gene or chromosome engineering to include variations thereof. In addition, starch derived from a plant grown from artificial mutations and variations of the above generic composition, which may be produced by known standard methods of mutation breeding, are also suitable herein.

Typical sources for the starches are cereals, tubers, roots, legumes and fruits. The native source can be corn, pea, potato, sweet potato, banana, barley, wheat, rice, sago, amaranth, tapioca, arrowroot, canna, sorghum, and waxy or high amylose varieties thereof. As used herein, the term “waxy” is intended to include a starch containing at least about 95% by weight amylopectin and the term “high amylose” is intended to include a starch containing at least about 40% by weight amylose.

The starch must be modified to achieve the desired film attributes. The film should be strong, yet appear flexible and should not appear brittle. It must be blocking and moisture resistant so that it does not adhere to itself, yet able to dissolve quickly when placed in the oral cavity, such as on the tongue.

Except in minor amounts, native starches are not suitable for the present invention without modification, and thus may be modified using any modification known in the art, including physical, chemical and/or enzymatic modifications, to obtain the desired film attributes.

Physically modified starches, such as sheared starches, or thermally-inhibited starches described in the family of patents represented by WO 95/04082, may be suitable for use herein.

Chemically modified products are also intended to be included as the base material and include, without limitation, those which have been crosslinked, acetylated and organically esterified, hydroxyethylated and hydroxypropylated, phosphorylated and inorganically esterified, cationic, anionic, nonionic, and zwitterionic, and succinate and substituted succinate derivatives thereof. Such modifications are known in the art, for example in Modified Starches: Properties and Uses, Ed. Wurzburg, CRC Press, Inc., Florida (1986).

Conversion products derived from any of the starches, including fluidity or thin-boiling starches prepared by oxidation, enzyme conversion, acid hydrolysis, heat and or acid dextrinization, thermal and or sheared products may also be useful herein.

Further suitable are pregelatinized starches which are known in the art and disclosed for example in U.S. Pat. Nos. 4,465,702, 5,037,929, 5,131,953, and 5,149,799. Conventional procedures for pregelatinizing starch are also known to those skilled in the art and described for example in Chapter XXII—“Production and Use of Pregelatinized Starch”, Starch: Chemistry and Technology, Vol. III—Industrial Aspects, R. L. Whistler and E. F. Paschall, Editors, Academic Press, New York 1967.

Any starch or starch blend having suitable properties, for use herein may be purified by any method known in the art to remove starch off flavors and colors that are native to the polysaccharide or created during processing. Suitable purification processes for treating starches are disclosed in the family of patents represented by EP 554 818 (Kasica, et al.). Alkali washing techniques, for starches intended for use in either granular or pregelatinized form, are also useful and described in the family of patents represented by U.S. Pat. Nos. 4,477,480 (Seidel) and 5,187,272 (Bertalan et al.).

Particularly suitable starches are starches capable of emulsifying or encapsulating the active ingredient so that there is no need for additional encapsulating or emulsifying agents. Such starches include, without limitation, hydroxyalkylated starches such as hydroxypropylated or hydroxyethylated starches, and succinated starches such as octenylsuccinated or dodecylsuccinated starches. The use of emulsifying or encapsulating starches are particularly useful in that a solution or dispersion of the film material (starch component, active agent, and optional additives) may be stored for later processing. The hydroxyalkylated starches have the added advantage of forming a softer film so that there is less or no need for a plasticizer.

To facilitate processing of the films, the starches are typically at least partially converted to reduce the viscosity and allow for the production of a high solids starch dispersion/solution, such as a 30% solids starch dispersion/solution. Particularly suitable starches are those with a viscosity of at least about 1000 cps at 20% solids and a viscosity of no more than about 10,000 cps at 90% solids.

Particularly suitable starches have a flow viscosity of at least about 7 seconds, more particularly at least about 10 seconds and no more than about 19 seconds, particularly no more than about 15 seconds. Flow viscosity, as used herein, is measured by the test defined in the Examples section, below.

The molecular weight of the starch is also important to its functionality in a film, particularly to film strength. For example, dextrins are not suitable in the present application.

The starch component may be a single modified starch, a blend of modified starches, or a blend of modified and native starches. Blends may be particularly useful to lower the cost of the film or to more easily achieve a variety of desirable properties and functionalities. If native starches are used, they may only be used in minor amounts, particularly less than 15%, more particularly less than about 10% by weight of the starch component.

The starch component may also comprise a cellulosic material or a gum, such as pullulan which is fully compatible and essentially substitutable for the starch. Other cellulosic materials and gums include without limitation carboxymethyl cellulose, hydroxypropyl cellulose, microcrystalline cellulose, ethylcellulose, cellulose acetate phthalate, hydrocolloids, carageenan, gums, and alginate. However, a cellulosic material or a gum is not an essential component of the film and may be used at levels of less than about 15 percent, more particularly less than about 10 percent by weight of the starch component, or may even be absent from the film. As starch is generally less expensive than pullulan, the cost of a pullulan film may be decreased by substituting starch for at least a portion of the pullulan, particularly at least about 50%, more particularly at least about 85%, most particularly at least about 90% of the pullulan by weight, without loss of the essential functionality of the pullulan film.

The starch component is typically used in amounts ranging from about 50 to about 100 percent, particularly from about 70 to 100 percent, most particularly from about 80 to 100 percent by weight of the film, exclusive of the active agent.

Active agents which may be delivered by the starch film include therapeutic, organoleptic, or cosmetic effect, such as breath fresheners, aromatizing agents, flavors, fragrances, vitamins, antiperspirants and deodorants, moisturizers, antiseptics, analgesics, sugars, perfumes, and pharmaceuticals. The active agent may be used at any amount desired, the only limitation being the potential load of the film. Typically, the amount of load will be depend upon the agent to be delivered and the intended use and will range from about 0.5 to about 40 percent, particularly about 0.5 to about 20 percent, more particularly about 0.5 to about 15 percent, by weight of the starch component. For example, a pharmaceutical agent will generally be delivered in substantially lower amounts than a breath freshener.

At least one plasticizer may be added to increase the apparent flexibility of the films. Further, a solid polyol plasticizer will generally provide better resistance to moisture absorption and blocking. One skilled in the art can chose a plasticizer to meet the desired needs of the film, such as chosing an edible plasticizer for an oral film. Plasticizers useful in the instant invention include, polyols, polycarboxylic acids, and polyesters. Examples of useful polyols include, but are not limited to ethylene glycol, propylene glycol, suger alcohols such as sorbitol, manitol, maltitol, lactitol; mono-, di-and oligosaccharides such as fructose, glucose, sucrose, maltose, lactose, and high fructose corn syrup solids and ascorbic acid. Examples of polycarboxylic acids include, but are not limited to, citric acid, maleic acid, succinic acid, polyacrylic acid, and polymaleic acid. Examples of polyesters include but are not limited to glycerin triacetate, acetylated-monoglyceride, diethyl phthalate, triethyl citrate, tributyl citrate, acetyl triethyl citrate, acetyltributyl citrate. The plasticizer may be present in any desired amount, particularly from 0 to about 15 percent, more particularly from 0 to about 10 by weight of the starch componenent.

Optional components may be added for a variety of reasons including without limitation, sweeteners, both natural and artificial; emulsifiers; humectants; surfactants; colorants; proteins such as gelatins; gums; flavors and flavor enhancers. Such optional components are typically added in minor amounts, particularly less than about 30% total by weight based upon the weight of the starch component.

The film may be made by a variety of processes known in the art. For example, the starch may be dispersed with the other film components in water or other solvent and dried into film form. In the alternative, the starch and other dry components may be blended and then dispersed with any additional film components in water or other solvent and dried into film form. Films may be formed from such dispersions or solutions by shaping it into a solidified form of a suitable thickness by any technique known in the art including, but not limited to, wet casting, freeze-drying, and extrusion molding. The dispersion or solution may also be directly coated or sprayed onto another edible product, such as a tablet or foodstuff, and dried to form an edible film.

A particularly suitable process for preparing the films of the present invention is by preparing a coating formulation by making a solution or dispersion of the film components, applying the mixture to a substrate, using knife, bar or extrusion die coating methods, drying the coated substrate to remove the majority of the solvent, and removing the film from the substrate. Suitable substrates include, but are not limited to, silicone elastomers, metal foils and metalized polyfoils, composite foils or films containing polyetrafluoroethylene materials or equivalents thereof, polyyether block amide copolymers, polyurethanes, polyvinylidene chloride, nylon, rubber-based polyisobutylene styrene, styrene-butadiene and styrene-isoprene copolymers, polyethylene, polyester, and other such materials useful in the art as releasable substrates.

The film is not completely dried in that some degree of water or other solvent remains. The amount of water may be controlled to obtain desired functionality. For example, more water typically results in a more flexible film, while too much water results in a film which will block and be tacky.

The film thickness will depend, in part, on the desired end use. Typically, the film thickness will be in the range of about 1 to 500 microns, particularly 25 to 100 microns. When prepared as an oral film for quick dissolution in the oral cavity, the film thickness is more preferably from about 25 to 50 microns.

The resultant films are light-weight and easy to carry. They are sufficiently strong and apparently flexible so as to be easily dispensable and handled.

The films exhibit moisture and blocking resistance, yet are quickly wetted when exposed to water, such as when placed on the tongue or other surface, followed by rapid dissolution and/or disintegration. The wettability and dissolution rates of the starches may be modified by one skilled in the art to target a specific delivery profile. For example, more rapid dissolution is typically preferred when the film is an oral film and particularly suitable films for such use are those which completely dissolve using the test described infra in less than about 30 seconds, particularly less than about 20 seconds, more particularly in less than about 10 seconds. For other uses, less rapid dissolution is necessary and films may completely dissolve in no more than about 60 seconds, particularly less than about 45 seconds, more particularly less than about 30 seconds.

One skilled in the art can also modify the film formulation to provide clarity and other desired characteristics by manipulation of the starch component and control of other components.

The films may be used for delivering any active agent for a variety of applications including personal care, skin care, wound care, pharmaceutical, and breath freshening. For example, the film may be used to deliver antiperspirant and/or deodorant to an underarm, perfume, vitamins or moisturizer to the skin, antiseptic to a wound, drugs, or aromatizing agents to the mouth.

### EXAMPLES

The following examples are presented to further illustrate and explain the present invention and should not be taken as limiting in any regard. All percents used are on a weight/weight basis.

In the examples below, the starches used are as follows:

Acetylated=acetylated (5% treatment) high amylose (70%) corn starch commercially available from National Starch and Chemical Company (Bridgewater, N.J., USA).

Converted=mannox converted waxy corn starch commercially available from National Starch and Chemical Company (Bridgewater, N.J., USA).

Corn=native corn starch commercially available from National Starch and Chemical Company (Bridgewater, N.J., USA).

OSA waxy 1=mannox degraded octenylsuccinated waxy corn starch commercially available from National Starch and Chemical Company (Bridgewater, N.J., USA).

PO waxy1=Hydroxypropylated (8.5% treatment) waxy corn starch with a water fluidity of 35* commercially available from National Starch and Chemical Company (Bridgewater, N.J., USA).

PO waxy2=Agglomerated hydroxypropylated (8.5% treatment) waxy corn starch with a water fluidity of 35* commercially available from National Starch and Chemical Company (Bridgewater, N.J., USA).

PO waxy3—Hydroxypropylated (8.5% treatment) waxy corn starch with a water fluidity of 15* commercially available from National Starch and Chemical Company (Bridgewater, N.J., USA).

Pullulan=pullulan (grade PF-20, molecular weight of 200,000) commercially available from Hayishibara Co., Ltd. (Japan).

Tapioca=native tapioca starch, commercially available from National Starch and Chemical Company (Bridgewater, N.J., USA).

*Water fluidity is an empirical measure of viscosity on a scale of 0-90, wherein fluidity is the reciprocal of viscosity. Water fluidity of starches is typically measured using a Thomas Rotational Shear-type Viscometer (commercially available from Arthur A. Thomas Co., Philadelphia, Pa.), standardized at 30° C. with a standard oil having a viscosity of 24.73 cps, which oil requires 23.12±0.05 sec for 100 revolutions. Accurate and reproducible measurements of water fluidity are obtained by determining the time which elapses for 100 revolutions at different solids levels depending on the starch's degree of conversion: as conversion increases, the viscosity decreases.

In the examples below, the procedures used are as follows:

Film casting—The films are casted using a knife-over-roll coating method, air dried overnight, and conditioned at 72° F. (22° C.) and 50% relative humidity.

Blocking resistance—Films are stacked on top of each other, conditioned for 24 hours at 104° F. (40° C.) and 75% relative humidity, then pulled apart to see whether or not they block (adhere).

Dissolution time—Dissolution of the films in the oral cavity are estimated by measuring the time, in seconds, that it takes for a square inch of film to disintegrate in a beaker of 98.6° F. (37° C.) water.

Flow Viscosity—Flow viscosity is measured as follows. The starch is slurried in water and jet cooked at 149° C. (300° F.) until fully gelatinized. The solids are adjusted to 5% (w/w). The temperature of the starch solution is controlled at 22° C. A total of 100 ml of the starch dispersion is measured into a graduated cylinder. It is then poured into a calibrated funnel while using a finger to close the orifice. A small amount is allowed to flow into the graduate to remove any trapped air, and the balance is poured back into the funnel. The graduated cylinder is then inverted over the funnel so that the contents draw (flow) into the funnel while the sample is running. Using a timer, the time required for the 100 ml sample to flow through the apex of the funnel is recorded.

The glass portion of the funnel is a standard 58°, thick-wall, resistance glass funnel whose top diameter is about 9 to 10 cm with the inside diameter of the stem being about 0.381 cm. The glass stem of the funnel is cut to an approximate length of 2.86 cm from the apex, carefully firepolished, and refitted with a long stainless steel tip which is 5.08 cm long with an outside diameter of 0.9525 cm. The interior diameter of the steel tip is 0.5951 cm at the upper end where it is attached to the glass stem; it is 0.4445 cm at the outflow end, with the restriction in the width occurring at about 2.54 cm from the ends. The steel tip is attached to the glass funnel by means of a Teflon tube. The funnel is calibrated so as to allow 100 ml of water to go through in 6 seconds using the above procedure.

### Example 1

### Comparison of Various Starches and Pullulan as Films

Films were made of a variety of starches or pullulan and the films were tested subjectively for flexibility, clarity, tack, blocking resistance and objectively for tensile strength and dissolution time. The results are shown in Table I, below.

TABLE I

| | Apparent | | | | Dissolution Time | Flow Viscosity |
|---|---|---|---|---|---|---|
| Starch | Flexibility | Clarity | Tack | Blocking | (sec) | (sec) |
| Pullulan | Flexible | Clear | None | None | 9 | — |
| PO waxy 1 | Flexible | Clear | None | None | 6.5 | 12.1 |
| Corn | Flexible | Hazy | None | None | >120 | 17.2 |
| | | | | | (did not dissolve) | |
| Tapioca | Flexible | Clear | None | None | 83 | 35.0 |
| Acetylated | Flexible | Hazy | None | None | >120 | 11.9 |
| PO waxy 3 | Flexible | Clear | None | None | 36 | 19.6 |
| OSA waxy 1 | Flexible | Clear | None | None | 42 | 10.1 |
| Converted | Flexible | Clear | None | None | 8.5 | 10.1 |
| PO Waxy 2: | Flexible | Clear | None | None | 13.5 | — |
| Pullulan 90:10 | | | | | | |
| PO waxy 2: | Flexible | Hazy | None | None | 7.5 | — |
| Corn 90:10 | | | | | | |

### Example 2

### Comparison of Film Formulations

Emulsions were made of a variety of starches and flavoring oil (orange oil). The emulsions were evaluated for stability at room temperature and the liquid was considered stable until phasing was visibly noticeable. The results are shown in Table II, below.

TABLE II

| | | | Liquid |
|---|---|---|---|
| | | | Stability |
| Starch | Orange Oil | Emulsified | (Days) |
| PO waxy 1 | 5% | Yes | 7 |
| PO waxy 1 | 10% | Yes | 7 |
| OSA waxy 1 | 5% | Yes | +30 |
| OSA waxy 1 | 10% | Yes | +30 |

We claim:

1. A composition comprising:
(a) a starch component comprising at least about 85 percent modified starch;
(b) an active agent; and
(c) 0 to about 15 percent of a plasticizer by weight of the starch component;
wherein such composition is a film.

2. The composition of claim 1, wherein the starch is selected from the group consisting of a hydroxyalkylated starch and a succinated starch.

3. The composition of claim 2, wherein the starch is a hydroxypropylated starch and there is no plasticizer.

4. The composition of claim 2, wherein the starch is a octenylsuccinated starch.

5. The composition of claim 1, wherein the starch component comprises 100 percent modified starch.

6. The composition of claim 1, wherein the starch component has a flow viscosity of from about 7 to about 19 seconds.

7. The composition of claim 6, wherein the starch component has a flow viscosity of from about 10 to about 15 seconds.

8. The composition of claim 1, wherein the plasticizer is selected from the group consisting of a polyol, a polycarboxyic acid and a polyester.

9. The composition of claim 8, wherein the plasticizer is selected from the group consisting of propylene glycol, sorbitol, mannitol, maltitol, lactitol, fructose, glucose, glycerin, sucrose, high fructose corn syrups, citric acid, and ascorbic acid.

10. The composition of claim 1, wherein the active agent is selected from the group consisting of breath fresheners, aromatizing agents, flavors, fragrances, vitamins, antiperspirants and deodorants, moisturizers, antiseptics, analgesics, sugars, perfumes, and pharmaceuticals

11. The composition of claim 1, further comprising at least one component selected from the group consisting of a sweetener, an emulsifier, a humectant, a surfactant, a protein, a gum, a colorant, and a flavor.

12. The composition of claim 1, wherein said film has a thickness of about 1 to about 500 microns.

13. The composition of claim 12, wherein the film has a thickness of about 25 to about 100 microns.

14. The composition of claim 13, wherein the film has a thickness of about 25 to about 50 microns.

15. The composition of claim 1, wherein the starch has been at least partially converted.

16. The composition of claim 1, wherein the film dissolves in water at physiological temperature in less than about 30 seconds.

17. The composition of claim 1, wherein the starch component comprises is a hydroxypropylated starch and comprises 80% of the film, and the active agent is a breath freshener and comprises 15% of the film.

18. A composition comprising pullulan, the improvement comprising substituting a starch component comprising at least about 85 percent modified starch for at least a portion of the pullulan in the composition.

19. A method of delivering an active agent to an animal comprising applying the composition of claim 1.

20. The method of claim 19, wherein the composition is used to deliver a therapeutic, organoleptic, or cosmetic effect
